(19) United States
(12) Patent Application Publication
(10) Pub. No.: US 2013/0303799 A1
(43) Pub. Date: Nov. 14, 2013

(54) PROCESS FOR THE PREPARATION OF POLY(ALKYLENE OXIDE) DERIVATIVES FOR MODIFICATION OF BIOLOGICALLY ACTIVE MOLECULES AND MATERIALS

(71) Applicants: Santiago Vaillard, Santa Fe (AR); Marianela Gonzalez, Santa Fe (AR)

(72) Inventors: Santiago Vaillard, Santa Fe (AR); Marianela Gonzalez, Santa Fe (AR); Ricardo José Antonio Grau, Santa Fe (AR)

(21) Appl. No.: 13/805,113

(22) PCT Filed: May 9, 2011
(86) PCT No.: PCT/US11/35733

§ 371 Date: Jul. 9, 2013

Related U.S. Application Data
(60) Provisional application No. 61/360,729, filed on Jul. 1, 2010.

(51) Int. Cl. A61K 47/48

(52) U.S. Cl. CPC A61K 47/48215
USPC 560/170

(57) ABSTRACT

A a method for producing activated linear polymers and activatable branched polymers thereof, is carried out by a) reacting a linear nonpeptidic activatable polymer, chemically blocked at one end, with an azole ring activating group that provides a leaving group to produce an intermediate polymer of the general formula poly-lm; b) reacting said poly-lm with an alkylating agent to form an imidazolium salt of the general formula poly-1m+(alkyl)X-; and c) reacting said poly-lm +(alkyl)X with a linker molecule bearing at least two nucleophilic moieties to produce an activatable branched polymer derivative thereof. In some embodiments “poly” is a polymer selected from the group consisting of poly(alkylene oxides), poly(oxyethylated polyols), poly(olefinic alcohols), and polymers of alkylene oxide and propylene oxide; in some embodiments “Im +” is an imidazolium ion; and in some embodiments “X-” is an anionic counterion.

### FIELD OF THE INVENTION

The present invention relates to an improved method for preparing poly(alkylene oxide) derivatives and related polymers for use in modifying the physicochemical properties of biologically active molecules and materials. More particularly, the present invention provides a new synthesis method for activated linear poly(ethylene glycol) which can be useful as is or as intermediates for the synthesis of activatable branched polymers thereof for preparing conjugates with biologically active materials, such as peptides, polypeptides, enzymes, proteins, oligonucleotides, and drug moieties. The improved process does not include the use of harmful reagents and discloses a convenient purification procedure for the branched polymer derivatives.

### BACKGROUND OF THE INVENTION

Some biologically active species used in the treatment of many diseases present diverse drawbacks that restrict their therapeutic efficacy. It is recognized that among the most critical issues limiting their performance are low stability and solubility in aqueous media, fast excretion rate, great susceptibility to enzymatic degradation, and/or undesirable immunological reactions. In the last few decades, significant efforts have been made to overcome these limitations and to improve the in vivo pharmacokinetic and pharmacodynamic performance of these biologically active species. Conjugation with polymers has proven to be an attractive and successful approach for this purpose.

A preferred class of polymers for preparing polymer conjugates are poly(alkylene oxides) (hereinafter PAO), such as poly(propylene glycol) and poly(ethylene glycol). Particularly, poly(ethylene glycol) (hereinafter PEG) has several properties that make it specifically suitable for conjugation, i.e., PEG is water-soluble, non-toxic and biocompatible. The PEG molecule can be structurally represented as
HO-(CH₂CH₂O)ₙCH₂CH₂-OH_{,}
   wherein n typically ranges from about 10 to about 2,000. Both terminal hydroxyl groups are rather non-reactive for covalent binding to biologically active moieties. Hence, PEG molecules generally must be activated or converted to more reactive polymers before they are suitable for conjugation.

The presence of two terminal hydroxyl groups per molecule of PEG leaves open the pathway to the production of di-activated PEG polymers, which in turn typically yields an undesirable high degree of cross-linking for biomaterial modification. PEG molecules with one blocked end group are therefore more suitable than PEG diol. For example, PEG molecules with a single non-reactive methyl end moiety, known as monomethoxy-poly(ethylene glycol) (hereinafter mPEG), are usually preferred as activatable polymers. The mPEG molecule can be structurally represented as having the formula
CHO-(CH₂CH₂O)ₙCH₂CH₂-OH,
   wherein the methyl group acts as a blocking group. The monofunctional mPEG can then be conveniently used to prepare activated linear mPEG derivatives having a single activation site. The monofunctional derivatives of interest can be structurally represented as having the formula
CH₃O-(CH₂CH₂O)ₙCH₂CH₂-Z,
   wherein Z is a reactive leaving group, selected from those well known in the art, for selectively attaching the polymer to a biologically active species.

Proteins and other biologically active materials only have a limited number of sites for the attachment of PEG. It is well known that such sites may also be involved in the biological activity. The epsilon-NH₂ moiety of lysine amino acids and the terminal amino groups are among the most common sites capable of reacting with activated polymers to yield polymer conjugates. Therefore, the conjugation reaction can be quite demanding because if an excessively high attachment of PEG derivatives is necessary to create a polymer cloud surrounding the biologically active material, the conjugate's biological activity could be negatively influenced. One approach to preparing polymer conjugates consists of reacting an activated linear PEG derivative with a biologically active material. There are some examples of commercial conjugates with linear PEG derivatives, such as Adagen®, Oncaspar®, Neulasta® and PegIntron®. In some cases, however, difficulties can arise when it is critical to minimize the loss of the biological activity. An approach to achieve an increased polymer cloud, but minimize the number of attachment sites, involves the use of activated branched polymers having two or more polymer chains per each linkage site. Branched mPEG derivatives are so far the most commonly used polymers, which can be structurally represented as having the formula

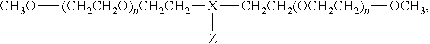

wherein X is a non-toxic and non-reactive moiety that links mPEG chains, which can have either the same or a different molecular weight. One particular example of Z is succinimidyl carbonate (Zalipsky et al., Biotechnol. Appl. Biochem. 1992; Miron and Wilchek, Bioconjug. Chem. 1993). This group has been widely used to couple PEG to biologically active materials reacting with the amino groups present, for example, in lysines of proteins and enzymes. The production of mPEG-disubstituted lysine activated as succinimidyl ester is well known in the art. One possibility is the reaction of branched PEG with N-hydroxysuccinimide and N,N-dicyclohexylcarbodiimide (Harris et al., U.S. Pat. No. 5,932,462).

In the polymer-conjugation field, more specifically in the field of PEGylation, it has been proven that modification with branched polymers is particularly efficient to improve the pharmacokinetic and pharmacodynamic properties of proteins (Interferon-α: Bailon et al., E.P. Patent No. 0 809 996; Lactoferrin: Nojima et al., Pham. Res, 2009), enzymes (Ribonuclease, Catalase, Asparaginase, Tripsin: Harris et al. U.S. Pat. No. 5,932,462), antibody fragments (Certolizumab pegol: Nesbitt et al. in PEGylated Protein Drugs: Basic Science and Clinical Application) and oligonucleotides (Pegaptanib, Ng et al. Nat. Rev. Drug Discov.). Accordingly, different processes for preparing branched activated PEGs have been claimed. For example, Martinez et al. (U.S. Pat. Nos. 5,643,575 and 5,919,455) have described several branched PEG molecules with different linking structures. Greenwald et al. (WO 98/41562) have synthesized a branched polymer with a central core of 1,3-diamino-2-propanol. Monfardini et al. (Bioconjugate Chem. 1995) have disclosed the preparation of a lysine-based branched polymer. Harris et al. (U.S. Pat. No. 5,932,462; U.S. Pat. No. 7,419,600) and Wu et al, (U.S. Pat. No. 7,365,127) have also described alternative methods of synthesizing the same lysine-based branched polymer.

Whereas the aforementioned methods provide the desired activated branched polymers, it is noticeable that toxic substances that are harmful to human health and the environment are currently used. In addition, very complex processes are typically required for preparing and/or purifying synthesis intermediates and/or final polymer derivatives. For example, U.S. Pat. Nos. 7,419,600 and 7,365,127 fall ill with some of these pointed out inconvenient features.

U.S. Pat. No. 7,419,600 describes two procedures for preparing branched mPEG derivatives having a lysine moiety as the linker. One of the disclosed preparation procedures is based on the use of an expensive commercially available activated mPEG, mPEG-p-nitrophenylcarbonate, which is synthesized from mPEG and p-nitrophenylchloroformate, the latter is a toxic compound. A further disadvantage of using p-nitrophenylcarbonate derivatives arises from the toxicity of the hydrophobic phenolic moiety and its high affinity for proteins. The other disclosed preparation procedure uses mPEG-succinimidyl carbonate, which is also commercially available but is an expensive compound. Moreover, the preparation of this activated species involves the use of phosgene, which is an extremely toxic gas.

U.S. Pat. No. 7,365,127 discloses another method for preparing the same branched mPEG derivatives linked with lysine, followed by conjugation to Interferon-β-1b. The synthetic procedure begins with the reaction of mPEG with triphosgene, a phosgene derivative considered slightly toxic since it is a solid crystal at room temperature. This solid compound, however, also involves careful handling. Moreover, a complex synthesis sequence comprising protection and de-protection of functional groups is typically needed to avoid the purification steps of synthesis intermediates and of the branched polymers. The purification steps are avoided at expense of increasing the number of synthesis steps, and of using several reagents successively in a very complex process that requires specialized chemical expertise for large scale manufacturing.

Thus, there is a need for improved procedures for preparing and purifying polymer derivatives for conjugation with the nucleophilic groups of biologically active moieties. The need for such improved procedures includes those that are more reliable, safer, faster, easily scalable, and/or more effective than the procedures currently available.

The present invention addresses previous shortcomings in the art by providing improved methods for preparing poly(alkylene oxide) derivatives and related polymers for use in modifying the physicochemical properties of biologically active molecules and materials.

### SUMMARY OF THE INVENTION

A first aspect of the present invention is a method for producing activated linear polymers and activatable branched polymers thereof, comprising:
a) reacting a linear nonpeptidic activatable polymer, chemically blocked at one end, with an azole ring activating group that provides a leaving group to produce an intermediate polymer of the general formula poly-Im;
b) reacting said poly-Im with an alkylating agent to form an imidazolium salt of the general formula poly-Im⁺(alkyl)X⁻; and
c) reacting said poly-Im⁺(alkyl)X⁻ with a linker molecule bearing at least two nucleophilic moieties to produce an activatable branched polymer derivative thereof, wherein:
poly is a polymer selected from the group consisting of poly(alkylene oxides), poly(oxyethylated polyols), poly(olefinic alcohols), and polymers of alkylene oxide and propylene oxide;
Im⁺ is an imidazolium ion; and

X⁻ is an anionic counterion selected from the group consisting of halides, nitrates, sulfonates, chlorates, citrates, succinates, tartrates, lactates, sulfates, phosphates, acetates, triflates, and borates.

The present invention provides the novel compound poly-Im⁺(alkyl)X⁻, which is useful as an activated linear polymer or for the synthesis of activatable branched polymers. The activated linear polymers and activatable branched polymers of the present invention may be used for the pegylation of biologically active materials, such as but not limited to peptides, polypeptides, enzymes, proteins, oligonucleotides, and drug moieties.

Another aspect of the present invention is a process for producing activated linear polymers and activatable branched polymers thereof, which does not comprise or involve the use of toxic substances, such as phosgene or triphosgene. Phosgene, triphosgene, and N,N-carbonyldiimidazole (CDI) are known to be carbonyl equivalents in some chemical reactions. (U.S. Pat. Nos. 5,359,086; 5,182,284, and 6,784,310). However, substitution of CDI, an azole ring activating group, with phosgene or triphosgene would not be applicable in the present invention. For instance, the reaction of mPEG with phosgene or triphosgene yields a chloroformate. Chloroformates are not useful for the synthesis of poly-Im⁺(alkyl)X⁻ derivatives (e.g., activatable branched polymers of the present invention) nor are they useful for pegylation due to their high reactivity and instability. It is well known in the art that to obtain a useful pegylating reagent, the chloroformate adduct must be transformed into a more stable, but still reactive intermediate, such as succinimidyl carbonate, benzotriazolate, or 2-nitrophenolate. Succinimidyl carbonate is most often used in the art, but is still not very stable, is highly reactive, and often yields high degrees of over-pegylation. Thus, phosgene and triphosgene are unable to act as substitutes for CDI in the processes of the present invention.

In certain aspects of the present invention a linear nonpeptidic activatable polymer is reacted with CDI to produce an intermediate polymer of the general formula poly-Im, wherein poly represents the polymer chain and Im is imidazole. The intermediate polymer of the general formula poly-Im is usually not very reactive and is particularly not as reactive as succinimidyl carbonate. The intermediate poly-Im can generally only be used in the pegylation of reactive amino groups (e.g., the ε amino group of lysine) and usually requires an undesirably high amount of starting material to be obtained. Thus, the low reactivity of poly-Im generally precludes its use in many pegylation reactions. In the processes of the present invention, poly-Im is converted into an imidazolium salt of the general formula poly-Im⁺(alkyl)X⁻. Poly-Im⁺(alkyl)X⁻ is a stable compound with a reactivity suitable for pegylation reactions. For instance, poly-Im⁺(alkyl)X⁻ is able to react with both the E and a amino groups of lysine. The reactivity of poly-Im⁺(alkyl)X⁻ was quite unexpected. This unexpected reactivity is particularly demonstrated in that the imidazolium salts of the present invention display an increased reactivity towards the more hindered and less reactive a amino group of lysine and other amino acids. The reactivity of poly-Im⁺(alkyl)X⁻ allows for good yields of bi-substitution of compounds, such as but not limited to, lysine. Such reactivity was quite unexpected and the discovery and process for preparing poly-Im⁺(alkyl)X⁻ are a major advancement in the field of pegylation. Thus, poly-Im⁺(alkyl)X⁻ is highly useful for pegylation and as a substitute for succinimidyl carbonate.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### DETAILED DESCRIPTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount or concentration (e.g., the molecular weight of a polymer) and the like, is meant to encompass variations of 20%, 10%, 5%, 1%, 0.5%, or even 0.1% of the specified amount.

One aspect of the present invention relates to an improved process for preparing new activated linear polymers that can be used as is or as synthesis intermediates for preparing activatable branched polymers thereof.

“Activated linear polymer” as used herein refers to a linear polymer prepared by the processes of the present invention that can be utilized as is to modify the physicochemical properties of biologically active molecules and materials. Alternatively, the activated linear polymer may be used to synthesize activatable branched polymers. “Activatable linear polymers” and “activatable branched polymers” as used herein refer to polymers that need to be activated before they can be utilized to modify the physicochemical properties of biologically active molecules and materials. Activation of the activatable linear polymers is accomplished by the processes of the present invention to yield activated linear polymers. The activatable branched polymers of the present invention are prepared by processes of the present invention from the activated linear polymers and can be activated by the processes of the present invention or by any methods known by those skilled in the art.

Exemplary activatable linear polymers that can be used to prepare the activated linear polymers and the activatable branched polymer derivatives of the present invention include, but are not limited to, poly(alkylene oxides) (PAO) such as polypropylene glycol) and poly(ethylene glycol); poly(oxyethylated polyols); poly(olefinic alcohols); and polymers of alkylene oxide and propylene oxide. In some embodiments of the present invention the activatable linear polymer is poly(ethylene glycol) (PEG). In other embodiments of the present invention the activatable linear polymer has one end group blocked, such as but not limited to monomethoxypoly(ethylene glycol) (mPEG). The activatable linear polymers of the present invention may have a molecular weight from about 100 to about 100,000 Da, from about 5,000 to about 75,000 Da, or from about 20,000 to about 50,000 Da.

Both the linear and branched polymer derivatives of the present invention are useful for modifying the physicochemical properties of biologically active molecules and materials such as, but not limited to, peptides, polypeptides, proteins, enzymes, oligonucleotides, and drug moieties. The present invention includes, but is not limited to, the synthesis of new activated linear PEGs, which are characterized as stable, easy to handle, non-toxic, and/or as having a reactivity similar to that shown by other derivatives well known in the art that are suitable for pegylation reactions. Another aspect of the present invention is activatable branched PEGs useful for conjugation reactions that are prepared efficiently and/or securely from the new activated linear PEGs, as described herein. In some embodiments of the present invention the activatable branched PEG polymer is (PEG)2Lys, wherein lysine is used as a linker molecule. The improved process of the present invention also includes a convenient purification method for the desired branched polymers from the crude of reaction mixture. The purification method comprises single or combined processes of membrane ultrafiltration and/or column chromatography.

The synthesis method of the present invention comprises few reaction steps. In one aspect of the present invention, only two reaction steps are involved when synthesizing the activated linear polymers from suitable activatable polymers. Suitable commercially available polymers include, but are not limited to, PAO or PEG polymers with one blocked end group, such as but not limited to mPEG. The first key step in the process of the present invention involves the reaction of an activatable linear polymer with an azole ring activating group. Azole ring activating groups are well known in the art and include those that can be activated by alkylation, such as but not limited to N,N-carbonyldiazoles. Exemplary azole ring activating groups include, but are not limited to, N,N-carbonyldiimidazole (CDI), N,N-carbonylbisbenzimidazole, N,N-thiocarbonylbisimidazole, and N,N-thiocarbonylbisbenzimidazole. In certain embodiments of the present invention, the reaction of the activatable linear polymer with the azole ring activating group provides a leaving group to yield an intermediate polymer of the general formula
poly-Im,
   wherein poly represents the polymer chain and Im is imidazole. In some embodiments the activatable linear polymer is a linear nonpeptidic activatable polymer. In certain embodiments of the present invention the activatable linear polymer is reacted with the azole ring activating group N,N-carbonyldiimidazole (CDI) to yield poly-Im. The use of CDI in some embodiments of the present invention presents advantages over other reagents used in the prior art, such as phosgene and triphosgene, since CDI is easier to manipulate and much less toxic than the activating compounds utilized in the prior art (e.g., phosgene and triphosgene).

As stated above, suitable activatable linear polymers include, but are not limited to, poly(alkylene oxides) such as polypropylene glycol) and poly(ethylene glycol); poly(oxyethylated polyols); poly(olefinic alcohols); and polymers of alkylene oxide and propylene oxide. In some embodiments of the present invention the activatable linear polymer is poly(ethylene glycol) (PEG). In other embodiments of the present invention the activatable linear polymer has one end group blocked, such as but not limited to monomethoxy-poly(ethylene glycol) (mPEG). In certain embodiments of the present invention the linear nonpeptidic activatable polymer is mPEG with a molecular weight from about 100 to about 100,000 Da.

The polymer activation reaction with the azole ring activating group is carried out by stirring at temperatures ranging from about 5° C. to about 80° C., from about 40° C. to about 70° C., or at about 60° C., for a period of about 12 to about 72 hours. The solvent used as reaction medium is chosen from those known in the art to perform this type of reaction. A non-limiting list of solvents in which the activatable polymer and azole ring activating group are soluble at room temperature or higher includes: halogenated solvents, linear oxygenated solvents, cyclic oxygenated solvents, polyoxygenated solvents, linear polyoxygenated solvents, and polar aprotic solvents. Specific exemplary solvents include, but are not limited to: methylene chloride, chloroform, acetonitrile (ACN) and other nitriles like propionitrile, tetrahidrofuran (THF), dioxanes, glycols, glymes, dimethyl sulfoxide (DMSO), dimethylformamide (DMF), 2-pyrrolidone, N-methyl-2-pyrrolidone (NMP), and mixtures thereof. In some embodiments of the present invention the solvent is THF. The formed product, poly-Im, is stable at room temperature or higher, typically up to about 100° C.

The second key reaction step in the process of the present invention comprises activation of the azole ring leaving group by alkylation. In some embodiments of the present invention the azole ring leaving group is imidazole (i.e., Im) and the Im group is activated by alkylation with a suitable alkylating reagent to form an imidazolium salt that can be structurally represented as
poly-Im⁺(alkyl)X⁻,
   wherein Im⁺ is the imidazolium ion, alkyl is an alkylic group, and X⁻ is an anionic counterion. “Alkyl” as used herein alone or as part of another group, refers to a straight, branched, or cyclic hydrocarbon containing from 1 to 20 carbon atoms. In some embodiments, the alkyl group may contain 1, 2, 3, 4, 5, 6, 7, 8, 9, 10, 11, 12, 13, 14, 15, 16, 17, 18, 19, or 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. The term “alkyl” is intended to include both substituted and unsubstituted alkyl unless otherwise indicated. Typical substituents include nonhydrogen atoms (e.g., halogens), functional groups (such as, but not limited to amino, sulfhydryl, carbonyl, hydroxyl, alkoxy, carboxyl, silyl, silyloxy, phosphate, and the like), hydrocarbyl groups, and hydrocarbyl groups substituted with one or more heteroatoms. Exemplary substituents include, but are not limited to, alkyl, lower alkyl, haloalkyl, alkenyl, alkynyl, cycloalkyl, cycloalkylalkyl, heterocyclo, heterocycloalkyl, aryl, arylalkyl, lower alkoxy, thioalkyl, hydroxyl, thio, mercapto, amino, imino, halo, cyano, nitro, nitroso, azido, carboxy, sulfide, sulfone, sulfoxy, phosphoryl, silyl, silyloxy, boronyl, and modified lower alkyl. “Anionic counterion” (i.e., X⁻) is an anion or an anionic group associated with the cationic charge of the imidazolium ion. Exemplary anionic counterions include, but are not limited to, halides such as chloride, iodide, fluoride, and bromide; nitrates; sulfonates such as C₁-C₆ alkyl sulfonates (e.g., methyl sulfonate, mesylate sulfonate, and orethyl sulfonate) and aryl sulfonates (e.g., benzene sulfonate and tosylates); chlorates; citrates; succinates; tartrates; lactates; sulfates such as alkyl sulfates (e.g., methyl sulfate and ethyl sulfate), arylsulfates (e.g., benzenesulfate and toluenesulfate), alkoxysulfates (e.g., methoxy sulfate and ethoxy sulfate), and aryloxysulfates; phosphates; acetates; triflates; and borates such as tetrafluoroborate.

Thus, the second step in some embodiments of the present invention comprises activating the Im group of poly-Im by alkylation with a suitable alkylating reagent to form the imidazolium salt poly-Im⁺(alkyl)X⁻.

A non-limiting list of suitable alkylating agents includes: alkyl halides, benzyl halides and related compounds, allyl halides, dialkylsulfates, alkyl, benzyl or allyl tosilates, mesilates or triflates, and related substituted compounds. In some embodiments of the present invention poly-Im is activated by an alkyl halide.

“Halide” as used herein refers to any suitable halogen, including Cl, Br and I. In certain embodiments of the present invention poly-Im is activated by alkyl iodides, such as but not limited to methyl iodide. In other embodiments of the present invention poly-Im comprises mPEG-Im and is activated by alkyl halide alkylation, and in certain embodiments mPEG-Im is activated by alkyl iodides, such as but not limited to methyl iodide.

The alkylation reaction of the azole ring, such as the Im ring of poly-Im polymers, is carried out under temperatures from about 5° C. to about 100° C., from about 10° C. to about 50° C., or at room temperature (about 25° C.), for a period of about 12 to about 24 hours. A non-limiting list of solvents that may be used to perform the alkylation reaction includes: halogenated solvents, linear oxygenated solvents, cyclic oxygenated solvents, polyoxygenated solvents, linear polyoxygenated solvents, polar aprotic solvents, and other solvents in which the activatable polymers are soluble at temperatures within the ranges quoted. In some embodiments of the present invention the alkylation reaction is carried out at room temperature. Specific exemplary solvents that may be utilized in the alkylation reaction include, but are not limited to: methylene chloride, chloroform, ACN and other nitriles like propionitrile, THF, dioxanes, glycols, glymes, DMSO, DMF, 2-pyrrolidone, NMP, and mixtures thereof. In some embodiments of the present invention the alkylation reaction is carried out in ACN.

The resulting activated linear polymers, e.g., poly-Im⁺(alkyl)X⁻, of the present invention, in some embodiments, are used to modify the physicochemical properties of biologically active materials and molecules such as, but not limited to, peptides, polypeptides, proteins, enzymes, oligonucleotides, and drug moieties. In other embodiments of the present invention the activated linear polymers are employed to prepare activatable branched polymers that are suitable for conjugation. The activatable branched polymers of the present invention can be activated for conjugation reactions by any method known by those skilled in the art.

In some embodiments the branched polymer derivatives of the present invention have a general structural formula that can be represented as

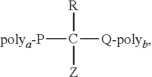

wherein polyₐ and poly_{b} are polymer chains from the activated linear polymers that may have the same or a different molecular weight, C denotes a carbon atom, R is a non-toxic and non-reactive moiety, P and Q are the same or different fragments capable of providing hydrolytically stable linkages, and Z is selected as to provide a functional group reactive toward or able to be activated to be reactive towards nucleophilic moieties of biologically active molecules and materials. The above formula is herein rewritten as polyₐ-P-C(R)(Z)-Q-poly_{b}.

In certain embodiments of the present invention, the synthesis method further comprises a third reaction step for preparing branched polymer derivatives by reacting the linear polymer derivatives, e.g., poly-Im⁺(alkyl)X⁻, resulting from the second reaction step with a linking molecule having at least two nucleophilic groups to produce an activatable branched polymer derivative thereof. Exemplary linking molecules include, but are not limited to, disubstituted alkyl diamines, triamines, and amino acids including natural and unnatural amino acid derivatives, diamino alkyls, dihydroxyalkyls, and dithioalkyls. Other exemplary linking molecules include, but are not limited to, lysine, lysine ester, and lysine ethyl ester. In some embodiments of the present invention the linking molecule is lysine. An exemplary synthesis procedure for lysine branched polymers, such as (poly)2Lys, involves reacting poly-Im⁺(alkyl)X⁻ with a lysine derivative soluble in an organic solvent. Lysine derivatives that are soluble in organic solvents include, but are not limited to, sylilated lysine derivatives. If such derivatives are not commercially available, the synthesis method additionally includes the well-known derivatization reaction of lysine with sylilated amides as an additional reaction step. An exemplary lysine branched polymer of the present invention is (mPEG)2Lys. In some embodiments of the present invention, the activatable branched polymer is mPEG-disubstituted lysine, i.e., (mPEG)2Lys.

As described above, the activatable branched polymers, in some embodiments, can be structurally represented as polyₐ-P-C(R)(Z)-Q-poly_{b}, wherein polyₐ and poly_{b} are polymer chains from the activated linear polymers, which may have the same or a different molecular weight, C represents a carbon atom, R is a non-reactive and non-toxic group, Z is a functional group capable of being activated to attach the branched polymer with biologically active compounds, and P and Q are the same or different fragments capable of providing hydrolytically stable linkages.

Exemplary linkages, P and Q, include but are not limited to amides, amines, ethers, carbamates (i.e., urethane linkages), urea, thiourea, thiocarbamates, thiocarbonates, thioethers, thioesters, and dithiocarbonate linkages. Ester linkages, which are hydrolytically unstable and potentially toxic aromatic moieties, should be avoided.

Exemplary functional groups for Z include, but are not limited to: 1) functional groups capable of reacting with an amino group, such as, but not limited to: a) carbonates, such as carbonates of p-nitrophenyl or succinimidyl; b) carbonyl imidazole; c) azlactones; d) cyclic imide thiones; and e) isocyanates or isothiocyanates; 2) functional groups capable of reacting with carboxylic acids or carboxylate groups and reactive carbonyl groups, such us, but not limited to: a) primary amines; and b) hidrazyne and hidrazyde functional groups, such as carbazates, semicarbamates, and thiocarbazates; 3) functional groups capable of reacting with mercapto and sulfhydryl moieties such as phenyl glyoxals; and 4) functional groups capable of reacting with hydroxyl groups such as carboxylic acids or other nucleophiles capable of reacting with an electrophilic center, such as but not limited to thiols, carboxylics, amines, hydroxyls, and active methylenes. The skilled artisan should recognize that Z encompasses the known activating moieties in pegylation chemistry and their conjugates. In some embodiments of the present invention the Z moiety also includes a spacer moiety situated proximal to the linker moiety.

In certain embodiments of the present invention the process further comprises the step of reacting poly-Im⁺(alkyl)X⁻ with a linker molecule bearing at least two nucleophilic moieties to produce an activatable branched polymer derivative thereof. The synthesis process of the present invention is useful for preparing branched polymer derivatives using a wide variety of linking molecules bearing at least two nucleophilic moieties. In some embodiments the linking molecules comprise one or more nucleophilic amino groups that are known to react promptly with the activated linear derivatives.

The synthesis procedure for preparing activatable branched polymer derivatives involves the reaction between the activated linear polymers, e.g., poly-Im⁺(alkyl)X⁻, and the linking molecule in organic solvents, at a suitable temperature and, in some embodiments, in the presence of a base. In some embodiments the linking molecule is lysine or a lysine derivative. In certain embodiments the lysine derivatives are sylilated derivatives. The sylilated lysine derivatives, in some embodiments, are obtained by the reaction of lysine with protection and/or derivatization reagents, such as but not limited to sylilated amides and halo-trialkylsilanes. A non-limiting list of useful reagents for preparing organic solvent soluble lysine derivatives includes: chlorotrimethylsilane, chloro-tert-butyldimethylsilane, and similar reagents. In some embodiments of the present invention the derivatization reaction comprises the reaction of lysine with sylilated amides. In other embodiments the derivatization reaction comprises the reaction of lysine with N,O-bis(trimethylsily)acetamide (BSA) under mild reaction conditions, as described herein below.

Reactions between the activated linear polymers, e.g., poly-Im⁺(alkyl)X⁻, and the linking molecule are carried out at temperatures ranging from about 5° C. to about 140° C., from about 30° C. to about 110° C., or at about 85° C., for a period of about 12 to about 36 hours. A non-limiting list of solvents that may be used to perform this reaction step includes: halogenated solvents, linear oxygenated solvents, cyclic oxygenated solvents, polyoxygenated solvents, linear polyoxygenated solvents, polar aprotic solvents, and other solvents in which both the activated alkylated polymers and the linker molecule are soluble at temperatures within the ranges quoted above. In some embodiments of the present invention the reaction is carried out at room temperature or higher. Specific exemplary solvents include, but are not limited to: methylene chloride, chloroform, ACN and other nitriles like propionitrile, THF, dioxanes, glycols, glymes, DMSO, DMF, NMP, water, 1,3-Dimethyl-3,4,5,6- tetrahydro-2(1H)-pyrimidinone (DMPU), and mixtures thereof. In some embodiments of the present invention the solvent is a mixture of organic solvents, such as but not limited to aprotic polar solvents with a high boiling point. In certain embodiments of the present invention the mixture comprises ACN and DMSO. Mixtures of solvents, such as but not limited to a mixture of an organic solvent and an aprotic polar solvent, range from about 0/100 to about 100/0, from about 25/75 to about 75/25, or are about 50/50.

The base optionally employed in the reaction of the activated linear polymers, e.g., poly-Im⁺(alkyl)X⁻, with the linker molecule may be selected from the ones usually utilized in this kind of transformation. Exemplary bases include, but are not limited to tertiary basic amines such as trialkylamines, dialkylarylamines, diarylalkylamines, and other amines like diazabicyclo-undecane and p-dimethylaminopyridine (DMPA); alkylic tertiary amines such as amines with aliphatic linear and branched groups like tributylamine, triethylamine, N,N-diisopropylethylamine; tetramethylguanidine; diethanolamine; and tetrabutylammonium hydroxide. In some embodiments of the present invention the base is N,N-diisopropylethylamine.

The branched polymer, in some embodiments, can be structurally represented as having the formula

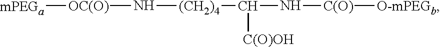

wherein mPEGₐ and mPEG_{b} have the structure: H₃CO-(CH₂CH₂O)ₙCH₂CH₂-, where n may be the same or different in mPEGₐ and mPEG_{b} and typically ranges from about 1 to about 1,500 to provide molecular weights of from about 100 to about 100,000 Da. The carboxyl group can be activated following diverse approaches to allow for coupling to biologically active materials, as is well known in the art.

Unlike prior art processes, the use of harmful or toxic compounds, such as phosgene or triphosgene (Harris et al., U.S. Pat. No. 7,419,600; Wu et al., U.S. Pat. No. 7,365,127) or p-nitrophenylcarbonate (Harris et al., U.S. Pat. No. 7,419,600) is avoided. Moreover, all the reaction steps are carried out easily and/or efficiently under mild conditions, without the need of intermediate steps for functional group protection, inactivation and/or purification. Consequently, the synthesis processes of the present invention can be performed under standard laboratory conditions. Additionally, the synthesis processes of the present invention are simple and require only standard chemical expertise, skills and/or labware.

Accordingly, one aspect of the present invention is a method for producing activated linear polymers and activatable branched polymers thereof, comprising:
a) reacting a linear nonpeptidic activatable polymer, chemically blocked at one end, with an azole ring activating group that provides a leaving group to produce an intermediate polymer of the general formula poly-Im;
b) reacting said poly-Im with an alkylating agent to form an imidazolium salt of the general formula poly-Im⁺(alkyl)X⁻; and
c) reacting said poly-Im⁺(alkyl)X⁻ with a linker molecule bearing at least two nucleophilic moieties to produce an activatable branched polymer derivative thereof,
   wherein:
poly is a polymer selected from the group consisting of poly(alkylene oxides), poly(oxyethylated polyols), poly(olefinic alcohols), and polymers of alkylene oxide and propylene oxide;
Im⁺ is an imidazolium ion; and
X⁻ is an anionic counterion selected from the group consisting of halides, nitrates, sulfonates, chlorates, citrates, succinates, tartrates, lactates, sulfates, phosphates, acetates, triflates, and borates.

Another aspect of the present invention is an efficient purification process for the desired polymer derivatives from the crude reaction mixtures. The disclosed method comprises, consists of, or consists essentially of one or both of the following purification steps: membrane filtration and/or column chromatography. In some embodiments of the present invention the membrane filtration process utilized is ultrafiltration and in certain embodiments the ultrafiltration process is operated under the diafiltration mode. Column chromatography may be based on affinity, ion exchange, size exclusion, hydrophobic interaction processes, and/or combinations thereof.

In some embodiments of the present invention the purification method comprises, consists of, or consists essentially of a two-step process, wherein the first step comprises diafiltration by tangential flow filtration to wash out low molecular weight species from the lumen solution while retaining the desired polymer derivatives. For the purposes of the present invention, diafiltration resulted to be an economic, simple, robust, fast and/or effective approach for removing low molecular weight impurities from the crude reaction mixtures. The resulting solution from the first purification step can be highly enriched in the desired branched derivative without significant losses in yields, which allows for a higher quantity of pure branched polymer to be loaded onto the chromatography column in the second and more expensive purification step without compromising the column capacity. In the second purification step, the resulting concentrate is highly purified by high-resolution chromatography. In some embodiments the resulting concentrate is purified by hydrophobic interaction chromatography. This procedure affords the desired branched polymers with high purity and high yields. The sequential two-step procedure has the advantage of high mass loading capability by virtue of which the column purification process can be more efficiently performed.

Diafiltration processing is performed in order to reduce as much low molecular weight species as possible for a given removed permeate volume and to minimize net losses of the desired polymer derivatives. For achievement of this goal, applicable materials and operating conditions depend upon the characteristics of the polymer derivatives as well as the specific purity requirements. The hydrophilic- hydrophobic properties and molecular weight cut-off of the membrane and the composition of the buffer solution are also chosen to make the process as cheap, flexible, robust, fast and efficient as possible, minimizing in turn membrane fouling processes. As known to persons having ordinary skill in the art, there is an ample range of materials and operating conditions for diafiltration processes. The mentioned materials and parameters are carefully set and controlled.

Exemplary purification conditions are provided below. In some embodiments of the present invention the purification process is utilized to purify mPEG-disubstituted lysine. In certain embodiments the mPEG-disubstituted lysine has PEG chains with a molecular weight of about 20,000 Da. A non-limiting list of useful synthetic polymeric membranes includes: polysulfones (PS), polyethersulfones (PES), polypropylene (PP) and polyvinylidene fluoride (PVDF). In some embodiments PES membranes with a molecular weight cut-off between about 20,000 and about 80,000 Da or between about 40,000 and about 60,000 Da are utilized. The pH, ionic strength and other operating conditions are adjusted in order to obtain good separation yields of the desired polymer derivative, such as but not limited to mPEG-disubstituted lysine, from the crude reaction mixture following aspects well known in the art. Ionic strength is controlled with inorganic salts, such as, but not limited to, sodium chloride. Concentrations of the inorganic salts range from about 0.00 to about 4.00 M, from about 0.01 to about 2.00 M, and from about 0.10 to about 0.30 M. Depending on the membrane characteristics, the ultrafiltration processes may be carried out at pressures up to about 315 kPa and at temperatures between about 4° C. to about 60° C. or at about room temperature. The separation process is carried out employing flow rates between about 15 to about 300 L m⁻²h⁻¹ or between about 60 to about 180 L m⁻²h⁻¹. In some embodiments of the present invention the flow rate is adjusted to about 90 Lm⁻²h⁻¹. After diafiltration processing, the purity of the resulting sample is greater than about 80%, as can be visually inferred from SDS-PAGE gels.

Column chromatography processing is ultimately performed to yield the desired polymer derivative with high purity. In some embodiments of the present invention column hydrophobic interaction chromatography is utilized. In certain embodiments column hydrophobic interaction chromatography is utilized after diafiltration processing. Unlike prior art approaches, highly enriched samples of the branched polymer can be loaded onto the column from the beginning, in such a way that there is a very high loading capability of the desired polymer derivative per batch operation; thus, making the global process faster and/or inexpensive. As known to one having ordinary skill in the art, there is a broad availability for selecting columns, mediums and operating conditions to perform hydrophobic interaction chromatography. The parameters, such as pressure, bed height, linear flow rate, medium composition, capacity and sample concentration are carefully set and controlled. A non limiting list of hydrophobic interaction chromatography mediums includes: butyl sepharose, octyl sepharose, phenyl sepharose, butyl agarose, hexyl agarose, octyl agarose, decyl agarose, and phenyl agarose. In certain embodiments of the present invention Phenyl Sepharose High Performance (GE Healthcare) is packed in a XK50 column (50 mm i.d., GE Healthcare) with a bed height of 12 cm and then equilibrated with NaCl 4M, and is used to separate the desired polymer derivative from the unreacted polymers, such as but not limited to separating the branched mPEG derivative from the unreacted mPEG and mPEG oligomers. The elution, in some embodiments, is performed using a stepwise gradient by ionic strength reduction. The purification processes of the present invention, in some embodiments, allow for the purification of great quantities of the desired polymer derivative, such as but not limited to mPEG-disubstituted lysine, without compromising the column capacity.

The present invention is explained in greater detail in the following non- limiting Examples.

### EXAMPLES

### Example 1

### Synthesis of mPEG(20 kDa)-OC(O)-Im (1)

Commercially available mPEG (20 kDa) (12.600 g, 6.3 10⁻⁴ mol) was dissolved in anhydrous THF (60 mL) at 60° C. CDI (0.293 g, 1.81 10⁻³ mol) was added and the solution was stirred at 60° C. for 18 hours. The solvent was removed under vacuum.

The residue was dissolved in water (100 mL) and then five-fold extracted with chloroform (5×100 mL). The organic phase was evaporated at reduced pressure and dried (5 mmHg) until constant weight. Yield: 97-99%. ¹H-RMN (300 MHz-Cl₃CD): 3.35 ppm (s, 3H, OMe); 3.60 ppm (brs, mPEG backbone); 4.43-4.52 ppm (m, superimposed on mPEG backbone peak, CH₂OC(O)); 7.04 ppm (s, 1H, Im-H); 7.40 ppm (s, 1H, Im-H); 8.11 ppm (s, 1H, Im-H).

### Example 2

### Synthesis of mPEG(20 kDa)-OC(O)-(ImMe)^{⊕}I^{⊖}(2)

The mPEG(20 kDa)-OC(O)-Im (2.000 g, 1 10⁻⁴ mol) obtained in Example 1 was dissolved in ACN (10 mL) at room temperature. Methyl iodide was added (1 mL, 1.6 10⁻² mol), and the solution was stirred at room temperature for 16 hours. The solvent was removed under reduced pressure and the resulting solid residue was dried (5 mmHg) until constant weight. Yield: 95-99%. ¹H-RMN (300 MHz-Cl₃CD): 3.36 ppm (s, 3H, OMe); 3.63 ppm (brs, mPEG backbone); 3.86 ppm (m, superimposed on mPEG backbone peak, CH₂OC(O)); 4.06 ppm (s, 3H, CH₃); 7.51 ppm (s, 2H, 2×Im-H); 9,96 ppm (s, 1H, Im-H).

### Example 3

### Synthesis of mPEG(20 KDa)-OC(O)-Lys-(O)CO-mPEG(20 KDa) (3)

a) Preparation of Me₃SiNH(CH₂)₄(COOSiMe₃)NHSiMe₃ solution: a solution of lysine (0.073 g, 0.5 mmol), BSA (0.65 mL, 2.62 10⁻² mol) and ACN (0.30 mL) was sonicated at room temperature until complete dissolution of the reagents.

b) mPEG(20 kDa)-OC(O)-(ImMe)^{⊕}I^{⊖} (1.931 g, 0.96 mmol) was dissolved in ACN (4 mL) and DMSO (4.00 mL) and then Me₃SiNH(CH₂)₄(COOSiMe₃)NHSiMe₃ solution (87.1 μL) and N,N-diisopropylethylamine (34.0 μL) were added. The molar relation mPEG-OC(O)-(ImMe)^{⊕}I^{⊖}: lysine was 2:1. The reaction mixture was stirred at 85° C. for 20 hours and allowed to reach room temperature. Brine (150 mL) was added and the aqueous phase was five-fold extracted with methylene chloride (40 mL each). The combined extract was evaporated and dried at reduced pressure (5 mmHg) until a constant weight. Yield: 95-99%. Reaction products were monitored using SDS-PAGE analysis. ¹H-RMN (300 MHz-Cl₃CD): 0.90-0.95 ppm (m, 2H, lysine backbone); 1.2-1.4 ppm (m, 6H, lysine backbone); 3.09 ppm (s, 3H, OMe); 4.14 ppm (m, 2H, CH₂OC(O)); 7.49 ppm (s, 1H, NH); 7.65 ppm (s,1H, NH).

### Example 4

### Purification of mPEG(20 kDa)-OC(O)-Lys-(O)CO-mPEG(20 kDa)

An aqueous solution containing 2.000 g of the solid obtained in example 3 was diafiltered through a 50,000 MW cutoff PES membrane (Vivaflow 200 cassette) using a 0.2 M NaCl buffer, pH 7, at a flow rate between 30 and 180 L m⁻²h⁻¹. After one ultrafiltration cycle with 4 L of 0.2 M NaCl buffer, the resulting solution was found to be highly enriched in the product of interest, i.e., mPEG(20 kDa)-OC(O)-Lys-(O)CO-mPEG(20 kDa) (purity >80%, as estimated by visual inspection of SDS-PAGE gel). The solution was five-fold extracted with methylene chloride (40 mL each). The combined extract was evaporated and dried with vacuum (5 mmHg). A sample of the dried product (0.6 g) was then dissolved in 4 M NaCl buffer (40 mL), and purified by hydrophobic interaction chromatography column (matrix: Phenyl Sepharose HP; column. XK 50/20. GE Healthcare). The purification process was conducted under conditions of stepwise gradient elution by ionic strength reduction. Eluting samples containing mPEG(20 kDa)-OC(O)-Lys-(O)CO-mPEG(20 kDa) were identified by SDS-PAGE analysis, pooled and four-fold extracted with methylene chloride (40 mL each). The combined extract was evaporated and dried at reduced pressure (5 mmHg). The solid obtained was analyzed by SDS-PAGE, MALDI TOF, ¹H-RMN and RP-HPLC-ELSD. These assays confirmed that mPEG(20 kDa)-OC(O)-Lys-(O)CO-mPEG(20 kDa) was obtained in good yield and with high purity as a result of the improved processes of the present invention.

### Example 5

### Activation of mPEG(20 kDa)-OC(O)-Lys-(O)CO-mPEG(20 kDa)

Activation of the branched polymer of the invention was carried out using techniques well known in the art. N-hydroxy succinimide (NHS) (4.83 mg, 0.042 mmol) was dissolved in 1 mL of anhydrous methylene chloride and 1 mL of anhydrous THF under nitrogen atmosphere and was kept under stirring at 0° C. in an ice bath. mPEG(20 kDa)-OC(O)-Lys-(O)CO-mPEG(20 kDa) (0.560 g, 0.014 mmol) obtained in Example 4 and N,N′-dicyclohexylcarbodiimide (DCC) (5.8 mg, 0.028 mmol) were added under nitrogen atmosphere. The solution was stirred 2 hours at 0° C. and then a new portion of DCC (2.9 mg, 0.014 mmol) was added. The reaction mixture was stirred for 30 minutes at 0° C. and then maintained for 16 hours at 4° C. without stirring. Dry diethyl ether (30 mL) was added and the precipitated product was separated by centrifugation. The solid was washed with dry diethyl ether, dried and re-dissolved in 2 mL of ACN. Acetic acid (35 μL) was added and the solution was kept 1 hour under stirring at room temperature. The upper phase was separated by centrifugation and the product was precipitated with dry diethyl ether, separated by centrifugation and once more washed and precipitated. The obtained solid was dried at reduced pressure (5 mmHg) until a constant weight. The conversion to mPEG(20 kDa)-OC(O)-Lys(mPEG(20 kDa) (O)CO)-OSu was evaluated using an spectrophotometric assay (Niemczyk and Van Arnum, 2008). Yield: 90-95%.

### Example 6

### Conjugation of IFN α-2a to mPEG(20 kDa)-OC(O)-Lys(mPEG(20 kDa)(O)CO)-OSu

The conjugation reaction was made using methodologies well known in the art. mPEG(20 kDa)-OC(O)-Lys(mPEG(20 kDa)(O)CO)-OSu (250 mg, 0.00625 mmol) was dissolved in 1 mM HCl previously cooled to 4° C. (2.5 mL), and kept with stirring in an ice bath. When the reagent was dissolved, IFN α-2a (14 mL of a 3.21 mg/ml solution, in 50 mM sodium-borate buffer pH 8.0) was quickly added, and the reaction was allowed to proceed for 3 hours at 4° C. under gentle stirring. The solution was quenched with acetic acid to pH 4.5, then diluted eight times with 10 mM NH₄Ac (pH 4.5) and loaded on ion exchange chromatography column (matrix: Fractogel EMD COO⁻ CM; column: XK 26/20, GE Healthcare; CV=60 mL; 4° C.). The purification process was conducted under conditions of stepwise gradient elution with the following buffers: 40 mM NH₄Ac pH 4.5; 0.12 M NaCl in 40 mM NH₄Ac pH 4.5; 0.5 M NaCl in 40 mM NH₄Ac pH 4.5 and 1 M NaCl in 40 mM NH₄Ac pH 4.5. Eluting samples were monitored by UV absorbance at 280 nm. Fractions containing the conjugate IFN α-2a—PEG2 were pooled and concentrated using Amicon® Ultra centrifugal filters (regenerated cellulose 10,000 MWCO) and analyzed. PEGylation crude mixture was characterized with SEC-HPLC (column: TSK Gel 3000 SW, 7.5×600 mm, Tosoh) indicating 36% yield of the conjugate IFN α-2a—PEG2, 2.5% of IFN α-2a-PEG2 oligomers, and 61.5% of unmodified IFN α-2a. SDS-PAGE gels (stained with Coomasie brilliant blue, and BaCl₂/I; using PEGASYS as conjugate of reference) was used in different stages of the process to confirm the presence of the conjugate. Concentration of proteins was determinates with UV absorbance at 280 nm, and with Lowry protein assay. Chromatographic yield: 72%. Final isolated yield: 26%.

The foregoing is illustrative of the present invention, and is not to be construed as limiting thereof. The invention is defined by the following claims, with equivalents of the claims to be included therein.

1. A method for producing activated linear polymers and activatable branched polymers thereof, comprising:
a) reacting a linear nonpeptidic activatable polymer, chemically blocked at one end, with an azole ring activating group that provides a leaving group to produce an intermediate polymer of the general formula poly-Im;
b) reacting said poly-Im with an alkylating agent to form an imidazolium salt of the general formula poly-Im⁺(alkyl)X⁻; and
c) reacting said poly-Im⁺(alkyl)X⁻ with a linker molecule bearing at least two nucleophilic moieties to produce an activatable branched polymer derivative thereof,
wherein:
poly is a polymer selected from the group consisting of poly(alkylene oxides), poly(oxyethylated polyols), poly(olefinic alcohols), and polymers of alkylene oxide and propylene oxide;
Im⁺ is an imidazolium ion; and
X⁻ is an anionic counterion selected from the group consisting of halides, nitrates, sulfonates, chlorates, citrates, succinates, tartrates, lactates, sulfates, phosphates, acetates, triflates, and borates.

2. The method of claim 1, further comprising the step of:
d) purifying said activatable branched polymer.

3. The method of claim 2, wherein the activatable branched polymer is purified by any of the following membrane filtration, column chromatography, or a combination thereof.

4. The method of claim 1, wherein said activatable polymer is a poly(alkylene oxide) having one blocked end group, said polymer having a molecular weight from about 100 up to about 100,000 Da.

5. The method of claim 4, wherein said poly(alkylene oxide) is polyethylene glycol.

6. The method of claim 5, wherein said polyethylene glycol is monomethoxy- poly(ethylene glycol).

7. The method of claim 6, wherein said monomethoxy-poly(ethylene glycol) has a molecular weight of about 10,000 to about 40,000 Da.

8. The method of claim 1, wherein said azole ring activating group is N,N-carbonyldiimidazole.

9. The method of claim 1, wherein said alkylating reagent is an alkyl halide.

10. The method of claim 9, wherein the alkyl halide is an alkyl iodide.

11. The method of claim 10, wherein the alkyl iodide is methyl iodide.

12. The method of claim 1, wherein said linker molecule is selected from the group consisting of disubstituted alkyl diamines, triamines, and amino acids.

13. The method of claim 12, wherein said linker molecule is a natural or unnatural amino acid derivative.

14. The method of claim 12, wherein said linker moiety is selected from the group consisting of lysine, diamino alkyls, dihydroxyalkyls, and dithioalkyls.

15. The method of claim 14, wherein the linker moiety is selected from the group consisting of lysine, lysine ester, and lysine ethyl ester.

16. The method of claim 1, wherein the branched polymer has a general structure:
mPEGₐ-O-C(O)-NH-(CH₂)₄-CH(C(O)OH)-NH-C(O)-O-mPEG_{b},
wherein:
mPEGₐ and mPEG_{b} have the structure H₃CO-(CH₂CH₂O)ₙCH₂CH₂-, where n may be the same or different in mPEGₐ and mPEG_{b}, and ranges from about 1 to about 1,500 to provide molecular weights from about 100 to about 100,000 Da;
and the carboxyl group can be further activated to allow coupling to biologically active molecules and materials.
